(19) United States
(12) Patent Application Publication
(10) Pub. No.: US 2020/0000843 A1
(43) Pub. Date: Jan. 2, 2020

(54) COMPOSITION FOR IMPROVEMENT OR PREVENTION OF PARKINSONIAN SYNDROME

(71) Applicant: MiZ Company Limited, Kanagawa (JP)

(72) Inventors: Fumitake Satoh, Kanagawa (JP); Shinichi Hirano, Kanagawa (JP); Ryosuke Kurokawa, Kanagawa (JP); Yi-Da Hsieh, Kanagawa (JP); Masatsugu Saito, Kanagawa (JP); Yusuke Ichikawa, Kanagawa (JP); Takeshirou Takekoshi, Niigata (JP)

(21) Appl. No.: 16/456,099

(22) Filed: Jun. 28, 2019

(30) Foreign Application Priority Data
Jun. 29, 2018 (JP) 2018-125228
Nov. 19, 2018 (JP) 2018-216264

(51) Int. Cl. A61K 33/00; A61P 25/16; A61P 25/14; A61K 9/00

(52) U.S. Cl. CPC A61K 33/00; A61K 9/007; A61P 25/14; A61P 25/16

(57) ABSTRACT

The present application provides: a composition for improving or preventing symptoms of human Parkinson's disease, multiple system atrophy or progressive supranuclear palsy, for example symptoms including at least tremors and/or forward-bending posture, or gait disorder, the composition including a hydrogen gas-containing gas as an effective ingredient; and a method for improving or preventing symptoms of Parkinson's disease, multiple system atrophy or progressive supranuclear palsy, which method comprises administering the composition to a human patient.

### FIELD OF THE INVENTION

The present invention relates to a composition for improving symptoms of Parkinson's disease (i.e., Parkinsonian syndrome), for example symptoms such as motor function disorders including tremors, forward-bending posture and gait disorder, in a human patient.

The present invention also relates to a method for improving or preventing the symptoms of Parkinson's disease in a human patient.

### BACKGROUND ART

Parkinson's disease is one of refractory neurodegenerative diseases, and is accompanied by a pathological change of dopaminergic neurons. Middle-aged or older persons have an increased risk of developing this disease, and examples of symptoms of the disease include slowing of movement (slowness of movement), shaking of limbs (tremors), hardening of muscle (muscle rigidity), and unstable postures in further advanced phases (Non-Patent Document 1).

Deficiency of dopamine in the brain, and oxidative stress causing a decrease in dopamine-secreting cells have been indicated as major causes of Parkinson's disease (Non-Patent Document 2). L-dopa preparations, dopamine receptor agonists and the like are clinically used for compensating for deficiency of dopamine. However, these pharmaceutical products have side effects such as nausea, sleepiness, orthostatic hypotension, swelling of feet and hallucinations.

Further, it has been pointed out that antioxidant therapy intended to prevent disruption of nerve cells by active oxygen may retard advancement of Parkinson's disease, and it has been reported that hydrogen water which is one of antioxidants may be useful for improvement of Parkinson's disease and reduction of loss of dopamine-secreting cells when administered to a human Parkinson's disease patient or a Parkinson's disease model mouse (Non-Patent Document 3, Non-Patent Document 4, and Non-Patent Document 5).

On the other hand, it has been reported that when a Parkinson's disease model rat intermittently breathed hydrogen gas (2%), advancement of symptoms was slightly lessened, and when the rat continuously breathed the hydrogen gas, the symptoms were advanced (Non-Patent Document 6).

### PRIOR ART LIST

### Non-Patent Document

[Non-Patent Document 1] Juntendo University Koshigaya Hospital (Saitama, Japan), Website, http://www.juntendo-koshigaya.jp/clinic/neurology/parkinson.html

[Non-Patent Document 2] Y. Fu et al., Neurosci. Lett. 2009; 453(2): 81-85

[Non-Patent Document 3] A. Yoritaka et al., BMC Neurology 2016; 16: 66

[Non-Patent Document 4] A. Yoritaka et al., Movement Disorders 2013; 28: 836-839

[Non-Patent Document 5] K. Fujita et al., PLoS ONE 2009; 4(9): e7247

[Non-Patent Document 6] M. Ito et al., Med Gas Res 2012; 2: 15

### SUMMARY OF THE INVENTION

### Technical Problem

It is an object of the present invention to provide a novel composition for improving or preventing symptoms of Parkinson's disease. The composition has few side effects, and can be conveniently produced.

A study was conducted in which a case where a Parkinson's disease model rat continuously or intermittently breathed hydrogen gas (2%) was compared with a case where the rat ingested hydrogen water. The result of the study showed that advancement of symptoms was lessened when the hydrogen water was ingested, and on the other hand, it was observed that advancement of the symptoms was slightly lessened when the hydrogen gas was intermittently breathed, and the symptoms were advanced when the hydrogen gas was continuously administered (M. Ito et al., Med Gas Res 2012; 2: 15).

Whether or not symptoms of Parkinson's disease are improved or prevented when a human Parkinson's disease patient inhales or breathes a hydrogen gas-containing gas is difficult to predict from the result of a study with an experimental animal as described above.

### Solution to Problem

The present inventors have extensively conducted studies, and resultantly found that unexpectedly, a hydrogen gas-containing gas improves or prevents specific symptoms of Parkinson's disease in a human patient.

Thus, the present invention includes the following features.

(1) A composition for improving or preventing symptoms of human Parkinson's disease, multiple system atrophy or progressive supranuclear palsy, the composition comprising a hydrogen gas-containing gas as an effective ingredient.

(2) The composition according to the above (1), wherein the symptoms of human Parkinson's disease, multiple system atrophy or progressive supranuclear palsy are symptoms including at least tremors and/or forward-bending posture, or gait disorder.

(3) The composition according to the above (1) or (2), wherein the multiple system atrophy is a disease selected from the group consisting of striatonigral degeneration, olivopontocerebellar atrophy and Shy-Drager syndrome.

(4) The composition according to any of the above (1) to (3), wherein a hydrogen concentration of the hydrogen gas-containing gas is 0.5 to 18.5% by volume.

(5) The composition according to any of the above (1) to (4), wherein the composition is administered to a patient by pulmonary administration.

(6) The composition according to any of the above (1) to (5), wherein the composition is prepared in situ using a hydrogen gas producing apparatus in administration of the composition to the patient.

(7) A method for improving or preventing symptoms of Parkinson's disease, multiple system atrophy or progressive supranuclear palsy in a human patient having Parkinson's disease, multiple system atrophy or progressive supranuclear palsy, the method comprising administering the composition according to any of the above (1) and (4) to (6) to the patient.

(8) The method according to the above (7), wherein the symptoms of human Parkinson's disease, multiple system atrophy or progressive supranuclear palsy are symptoms including at least tremors and/or forward-bending posture, or gait disorder.

(9) The method according to the above (7) or (8), wherein the multiple system atrophy is a disease selected from the group consisting of striatonigral degeneration, olivopontocerebellar atrophy and Shy-Drager syndrome.

### Effect of the Invention

According to the present invention, in a human patient having Parkinson's disease, multiple system atrophy or progressive supranuclear palsy, symptoms of the diseases, particularly symptoms of Parkinson's disease, for example symptoms including motor function disorders, for example at least tremors and/or forward-bending posture, or gait disorder can be markedly improved or prevented by inhalation or breathing of hydrogen gas.

### BRIEF DESCRIPTION OF THE DRAWINGS

FIGS. 1A to 1C show the results of measuring symptoms of a Parkinson's disease patient with scale values of 0 to 5.0 (a greater value indicates a worse condition) on a visual analog scale (VAS) over three months after the start of inhalation of hydrogen gas, where graphs A, B and C show improvements of the symptoms of action tremors, slowness of movement and tiny step gait, respectively; and

FIGS. 2A to 2F show the results of measuring symptoms of a Parkinson's disease patient with scale values of 0 to 5.0 (a greater value indicates a worse condition) on a visual analog scale (VAS) over three months after the start of inhalation of hydrogen gas, where graphs A, B and C show improvements of the symptoms of slowness of movement, forward-bending posture and rigidity, respectively, and graphs D, E and F show improvements of the symptoms of tiny step gait, bowel movement (constipation) and sleep (insomnia), respectively.

### MODES FOR CARRYING OUT THE INVENTION

The present invention will be described in further detail.

### 1. Parkinson's Disease, Multiple System Atrophy or Progressive Supranuclear Palsy

### (1) Parkinson's Disease

Parkinson's disease is one of refractory neurodegenerative diseases, and is a disease accompanied by a pathological change of dopaminergic neurons. Middle-aged or older persons have an increased risk of developing this disease, and examples of symptoms of the disease include slowing of movement (slowness of movement), shaking of limbs (tremors), hardening of muscle (muscle rigidity), and unstable postures in further advanced phases (Juntendo University Koshigaya Hospital (Saitama, Japan), Website, http://www.juntendo-koshigaya.jp/clinic/neurology/parkinson.html).

In diagnosis of symptoms of a Parkinson's disease patient, and the therapeutic efficacy, the symptoms of the patient are evaluated on the basis of, for example, a scale on Parkinson's disease UPDRS (Unified Parkinson's Disease Rating Scale). The scale on UPDRS consists of four parts (that is, parts Ito IV). Evaluation is performed for mental function, behavior and temper in part I, daily life action in part II, motor function in part III, and complications from therapy in part IV. Some examples of symptoms related to motor function are shown below.

The term “resting tremors” is a symptom in which fingers or legs shake at rest.

The term “tremors in hand motion or posture correction” is a shake occurring in motion or posture correction.

The term “slowness of movement” is a symptom in which a motion cannot be started quickly, and movement is slowed even after the motion is started.

The term “rigidity” is an increase in muscle tone at the time of subjecting the joints of the four limbs or the trunk to passive bending exercises at rest. This condition is also referred to as muscle rigidity.

The term “forward-bending posture” is a condition in which the waist is bent in a dogleg shape.

The term “festinating gait” is a symptom in which when waking is started, forward-bending posture is intensified, so that walking cannot be stopped.

The term “pulsion” is a phenomenon in which forward, backward or lateral pushing causes a dash in the direction of the pushing.

The term “tiny step gait” is continuous walking with a small and almost constant step length.

Herein, the symptoms of Parkinson's disease (sometimes referred to as “Parkinson's disease symptoms”) which are improved or prevented by the present invention are symptoms attributed to Parkinson's disease and described in, for example, the scale of UPDRS, and symptoms similar to the symptoms of Parkinson's disease patients, preferably symptoms related to motor function, for example symptoms such as tremors, forward-bending posture, slowness of movement and gait disorder, more preferably symptoms including tremors and/or forward-bending posture, or gait disorder.

### (2) Multiple System Atrophy

The multiple system atrophy is a collective designation of diseases including striatonigral degeneration, olivopontocerebellar atrophy and Shy-Drager syndrome, all of which are accompanied by Parkinson's disease symptoms, are of unexplained origin, and are designated as intractable diseases in Japan. Hereinafter, these diseases will be described on the basis of information provided by Intractable Disease Information Center (Japan).

The striatonigral degeneration is a disease observed in about 30% of patients of multiple system atrophy. Like Parkinson's disease, the striatonigral degeneration causes poor expression, hardened and stiffened muscle, and slow or slowed movement. In addition, the striatonigral degeneration causes difficulty of talking, destabilized standing and walking, and increased susceptibility to falling down. It is observed that hands and fingers occasionally shake. Eventually, lightheadedness upon standing, autonomic nervous symptoms such as difficulty of urinary drainage or getting constipated, or unsteadiness or difficulty of talking resulting from damage to the cerebellum is observed, but there is little evident intelligence disorder.

The olivopontocerebellar atrophy is developed with a cerebellar symptom such as unsteadiness in standing and walking, and progressed with the cerebellar symptom as a main symptom. The olivopontocerebellar atrophy is a disease observed in about 70 to 80% of patients of multiple system atrophy. The symptoms of the disease include unsteadiness in standing and walking, impairment of voice pronouncement, and impairment of minute and precise movement of hands. Motions which are naturally made without thinking every day, such as handling chopsticks, fastening a button, unfastening a button, writing letters, and pulling on trousers while standing, cannot be smoothly performed. The olivopontocerebellar atrophy may be accompanied by an autonomic disorder or a Parkinson's disease symptom when advanced.

The Shy-Drager syndrome is developed with an autonomic disorder that causes urinary incontinence or fainting, and since there is no difference between the Shy-Drager syndrome and the olivopontocerebellar atrophy or striatonigral degeneration in histopathological findings, the Shy-Drager syndrome is considered to be the same disease as the olivopontocerebellar atrophy or striatonigral degeneration. Patients of Shy-Drager syndrome are presumed to occupy 16% of patients of multiple system atrophy. In this disease, a cerebellar disorder or a Parkinson's disease symptom is developed subsequently to the autonomic disorder.

### (3) Progressive Supranuclear Palsy

The progressive supranuclear palsy (PSP) is a disease which is developed in middle-aged or older persons and in which nerve cells of globus pallidus, subthalamic nuclei, cerebellar dentate nuclei, red nuclei, substantia nigra and brainstem tegmentum fall, and abnormally phosphorylated tau protein is accumulated in nerve cells and glia cells. Pathologically, tuft of abnormal fibers (tufted astrocytes) in astrocytes are considered to be findings specific to PSP. PSP is characterized neurologically by susceptibility to falling down, supranuclear gaze palsy, Parkinsonism, dementia and the like. The cause of development is unknown. Males have an increased risk of developing PSP. PSP is similar in initial symptoms to Parkinson's disease, but rarely causes resting tremors, and noticeably causes susceptibility to falling down in walking, freezing of gait, and postural maintenance disorder. With the advancement of PSP, backward bending and warped posture of the neck, vertical supranuclear eyeball movement disorder (voluntary vertical movement of eyeball movement is slowed in the early stage, and it becomes eventually impossible to gaze downward), articulation disorder or swallowing disorder, and dementia or impaired attention characterized by remembrance disorder and slowness of thinking occur. Gait inability and standing inability gradually progress, resulting in being confined to bed.

2. Composition for Improving or Preventing Symptoms of Parkinson's Disease, which Includes Hydrogen Gas-Containing Gas

A first aspect of the present invention provides a composition for improving or preventing, in a human patient having Parkinson's disease, multiple system atrophy or progressive supranuclear palsy, symptoms caused by the disease, particularly symptoms of Parkinson's disease, for example symptoms including motor function disorders, for example at least tremors and/or forward-bending posture, or gait disorder, the composition including a hydrogen gas-containing gas as an effective ingredient.

Herein, the term “hydrogen” which is an effective ingredient of the composition of the present invention is molecular hydrogen (that is, gaseous hydrogen), and is referred to simply as “hydrogen” or “hydrogen gas” unless otherwise specified. In addition, the term “hydrogen” as used herein refers to hydrogen represented by the molecular formula of H₂, D₂ (deuterium) or HD (hydrogen deuteride), or a mixed gas thereof. D₂ is expensive, and is known to have a superoxide scavenging effect higher than that of H₂. The hydrogen usable in the present invention is H₂, D₂ (deuterium), HD (hydrogen deuteride), or a mixed gas thereof, preferably H₂. Alternatively, D₂ and/or HD may be used in place of H₂ or in combination with H₂.

The hydrogen gas-containing gas is preferably air containing hydrogen gas, or a mixed gas containing hydrogen gas and oxygen gas. The concentration of hydrogen gas in the hydrogen gas-containing gas is more than zero (0) and not more than 18.5% by volume, for example 0.5 to 18.5% by volume, preferably 1 to 10% by volume, for example 2 to 10% by volume, 2 to 8% by volume, 3 to 10% by volume, 3 to 8% by volume, 3 to 7% by volume, 3 to 6% by volume, 4 to 10% by volume, 4 to 8% by volume, 4 to 7% by volume, 4 to 6% by volume, 4 to 5% by volume, 5 to 10% by volume, 5 to 8% by volume, 6 to 10% by volume, 6 to 8% by volume or 6 to 7% by volume, more preferably 5 to 10% by volume or 5 to 8% by volume, for example 6 to 8% by volume or 6 to 7% by volume. In the present invention, the improvement or prevention effect on symptoms of Parkinson's disease tends to be enhanced as the hydrogen gas concentration increases (for example 5 to 10% by volume) below the explosion limit. In addition to this, further, in the present invention, the improvement or prevention effect on symptoms of Parkinson's disease tends to be enhanced as the period of time during which the hydrogen gas-containing gas is inhaled or breathed per day increases (for example about 90 to 180 minutes or more).

Hydrogen is a combustible and explosive gas, and therefore in improvement or prevention of symptoms of Parkinson's disease, it is preferable to administer the composition to a human patient having Parkinson's disease, multiple system atrophy (for example a disease selected from the group consisting of striatonigral degeneration, olivopontocerebellar atrophy and Shy-Drager syndrome) or progressive supranuclear palsy with the hydrogen incorporated in the composition of the present invention under safe conditions.

When the gas other than hydrogen gas is air, the concentration of the air is in the range of, for example, 81.5 to 99.5% by volume.

When the gas other than hydrogen gas is a gas containing oxygen gas, the concentration of the oxygen gas is in the range of, for example, 21 to 99.5% by volume.

Nitrogen gas may be present as another main gas. A gas such as carbon dioxide which is a gas present in the air may be present in an amount equivalent to the abundance in the air.

In the present invention, a hydrogen-dissolved liquid can be administered to or ingested in a human patient having Parkinson's disease, multiple system atrophy or progressive supranuclear palsy in combination with administration of the hydrogen gas-containing gas as necessary.

When the hydrogen gas-containing gas is administered in combination with a hydrogen-dissolved liquid, the composition of the present invention can be administered before administration of the hydrogen-dissolved liquid, in parallel to administration of the hydrogen-dissolved liquid or after administration of the hydrogen-dissolved liquid.

The hydrogen-dissolved liquid is specifically an aqueous liquid in which hydrogen gas is dissolved, and here, examples of the aqueous liquid include, but are not limited to, water (for example sterilized water and purified water), physiological saline, buffer solutions (for example, buffer solutions having a pH of 4 o 7.4), ethanol-containing water (for example, ethanol content: 0.1 to 2% by volume), drip-feed solutions, infusion solutions, injection solutions and beverages. The hydrogen concentration of the hydrogen-dissolved liquid is, for example, 1 to 10 ppm or more, preferably 1.2 to 8 ppm, for example 1.5 to 7 ppm, 1.5 to 5 ppm, 2 to 10 ppm, 2 to 8 ppm, 2 to 7 ppm, 2 to 6 ppm, 2 to 5 ppm, 3 to 10 ppm, 3 to 8 ppm, 3 to 7 ppm, 4 to 10 ppm, 4 to 8 ppm, 5 to 8 ppm, 5 to 10 ppm, 7 to 10 ppm, more preferably 3 to 10 ppm, for example 3 to 7 ppm, 3 to 8 ppm, 4 to 8 ppm, 5 to 8 ppm, 5 to 10 ppm or 7 to 10 ppm. In the present invention, the improvement or prevention effect on symptoms of Parkinson's disease tends to be enhanced as the hydrogen gas concentration increases below the explosion limit. The patient can ingest, for example, 0.5 to 2.0 L or more per day of hydrogen-dissolved water having a hydrogen gas concentration of 5 to 10 ppm.

A pharmaceutical product for treating symptoms of Parkinson's disease may be added to the hydrogen-dissolved liquid. Alternatively, the pharmaceutical product may be administered separately from administration of the hydrogen-dissolved liquid or the hydrogen gas-containing gas. Examples of the pharmaceutical product include, but are not limited to, levodopa preparations, dopamine receptor agonists, MAO B inhibitors and amantadine hydrochloride.

The hydrogen gas-containing gas or the hydrogen-dissolved liquid is formulated to a predetermined hydrogen gas concentration, and then charged into, for example, a pressure-resistant vessel (for example, a stainless cylinder, an aluminum can, preferably a pressure-resistant plastic bottle with its inside laminated with an aluminum film (for example pressure-resistant PET bottle) and a plastic bag, an aluminum bag). Aluminum has a property of being hardly permeable to hydrogen molecules. Alternatively, the hydrogen gas-containing gas or the hydrogen-dissolved liquid may be prepared in situ using an apparatus such as a hydrogen gas producing apparatus, a hydrogen water producing apparatus or a hydrogen gas adding apparatus, for example a known or commercially available hydrogen gas supplying apparatus (an apparatus for production of a hydrogen gas-containing gas), a hydrogen adding device (an apparatus for production of hydrogen water) or a non-destructive hydrogen incorporating device (for example an apparatus for non-destructively adding hydrogen gas to the inside of a bag for a biologically applicable liquid such as a drip-feed solution) in administration.

The hydrogen gas supplying apparatus ensures that hydrogen gas generated by reaction of a hydrogen generator (for example metallic aluminum or magnesium hydride) with water can be mixed with a diluting gas (for example air or oxygen) at a predetermined ratio (Japanese Patent No. 5228142). Alternatively, hydrogen gas generated by employing electrolysis of water is mixed with a diluting gas such as oxygen or air (Japanese Patent No. 5502973, Japanese Patent No. 5900688 or the like). In this way, a hydrogen gas-containing gas having a hydrogen concentration within the range of 0.5 to 18.5% by volume can be prepared.

The hydrogen adding device is an apparatus in which hydrogen is generated using a hydrogen generator and a pH adjustor, and dissolved in a biologically applicable liquid such as water (Japanese Patent No. 4756102, Japanese Patent No. 4652479, Japanese Patent No. 4950352, Japanese Patent No. 6159462, Japanese Patent No. 6170605, Japanese Patent Laid-Open No. 2017-104842 or the like). The combination of a hydrogen generator and a pH adjustor is, for example, a combination of metallic magnesium and a strongly acidic ion-exchange resin or an organic acid (for example malic acid or citric acid), a combination of metallic aluminum powder and calcium hydroxide powder. In this way, a hydrogen-dissolved liquid having a dissolved hydrogen concentration of about 1 to 10 ppm can be prepared (for example, an ultrahigh-concentration hydrogen water production kit (7 ppm or 10 ppm), trade name “7 Water” (QUASIA, Osaka, Japan), “7 WATER”, or the like).

The non-destructive hydrogen incorporating device is an apparatus or device in which hydrogen molecules are added to a commercially available biologically applicable liquid (for example, encapsulated in a hydrogen-permeable plastic bag such as a polyethylene bag) such as a drip-feed solution from the outside of a package, and such an apparatus or device is commercially available from, for example, MiZ Company Limited (Kanagawa, Japan) (http://www.e-miz.co.jp/technology.html). In this apparatus, a bag containing a biologically applicable liquid is immersed in saturated hydrogen water so that the bag is permeated with hydrogen, whereby hydrogen can be aseptically dissolved in the biologically applicable liquid until reaching equilibrium. The apparatus includes, for example, an electrolytic bath and a water bath, and water in the water bath is circulated through the electrolytic bath and the water bath, so that hydrogen can be produced by electrolysis. Alternatively, a simplified disposable device can be used for the same purpose (Japanese Patent Laid-Open No. 2016-112562, or the like). This device includes a biologically applicable liquid-containing plastic bag (a hydrogen-permeable bag, for example a polyethylene bag) and a hydrogen generator (for example metallic calcium, metallic magnesium/cation-exchange resin or the like) in an aluminum bag, and the hydrogen generator is covered with, for example, a nonwoven fabric (for example water vapor-permeable nonwoven fabric). The hydrogen generator covered with a nonwoven fabric is wetted with a small amount of water such as water vapor to generate hydrogen, and the plastic bag is permeated with the hydrogen and the hydrogen is non-destructively and aseptically dissolved in the biologically applicable liquid.

A hydrogen gas-containing gas or a hydrogen saturated biologically applicable liquid (for example sterilized water, physiological saline, or drip-feed solution), which is prepared using the above-described apparatus or device, can be orally or parenterally administered to a human patient having Parkinson's disease, multiple system atrophy or progressive supranuclear palsy.

Another form of the composition of the present invention includes a hydrogen generator-containing dosage form (for example, a tablet or a capsule) which is prepared so as to be orally administered to (or ingested in) a human patient having Parkinson's disease, multiple system atrophy or progressive supranuclear palsy and which enables hydrogen to be generated in the gastrointestinal tract. Preferably, the hydrogen generator is constituted by components approved as, for example, food or food additives.

### 3. Improvement or Prevention of Symptoms of Parkinson's Disease

A second aspect of the present invention provides a method for improving or preventing symptoms of human Parkinson's disease, multiple system atrophy or progressive supranuclear palsy, particularly symptoms of Parkinson's disease, for example symptoms including motor function disorders caused by the diseases, for example at least tremors and/or forward-bending posture, or gait disorder, in a patient having the disease, the method comprising administering the composition of the present invention to the patient.

The composition of the present invention enables marked improvement of QOL (quality of life) of a patient. The Parkinson's disease symptoms are markedly improved by breathing or inhalation of a hydrogen gas-containing gas as described later in Examples. This also indicates that Parkinson's disease symptoms can be prevented by continuously performing treatment by breathing or inhaling the hydrogen gas-containing gas. Before or after administration of the composition of the present invention, a hydrogen-dissolved liquid may be administered to the patient in combination as necessary. The hydrogen concentration in the hydrogen-dissolved liquid, and the apparatus or device for preparing the hydrogen-dissolved liquid are as described above.

The method for administering the composition of the present invention to a human patient having Parkinson's disease, multiple system atrophy or progressive supranuclear palsy is preferably pulmonary administration by, for example, inhalation or breathing when hydrogen gas is an effective ingredient. In inhalation of the gas, the gas can be inhaled from the mouth or the nose through a nasal canula or a mask-type device covering the mouth and the nose, sent to the lung, and delivered to all parts of the body through blood.

In addition, when the hydrogen-dissolved liquid is administered to a patient, oral administration, or intravenous administration or intraarterial administration (including drip infusion) is preferable. For the hydrogen-dissolved liquid to be orally administered, the liquid cooled by storing the liquid preferably at a low temperature, or the liquid stored at normal temperature may be administered to a human patient having Parkinson's disease, multiple system atrophy or progressive supranuclear palsy. It is known that hydrogen is soluble in water at a concentration of about 1.6 ppm (1.6 mg/L) at normal temperature and normal pressure, and the temperature-dependent variation of the solubility of hydrogen is relatively small. Alternatively, when the hydrogen-dissolved liquid is in the form of, for example, a hydrogen gas-containing drip-feed solution or injection solution prepared using the non-destructive hydrogen incorporating device, the hydrogen-dissolved liquid may be administered through parenteral administration such as intravenous administration or intraarterial administration to a human patient having Parkinson's disease, multiple system atrophy or progressive supranuclear palsy.

A hydrogen gas-containing gas having the above-described hydrogen concentration or a hydrogen-dissolved liquid having the above-described hydrogen concentration can be administered once or two or more times (for example two or three times) per day over a period of 1 week to three months or more, for example 1 week to 6 months or more, or 1 year to 3 years or more, to a human patient having Parkinson's disease, multiple system atrophy or progressive supranuclear palsy. When the hydrogen gas-containing gas is administered, the hydrogen gas-containing gas can be administered, for example, for 10 minutes to 2 hours or more, preferably 20 minutes to 40 minutes or more, still more preferably 30 minutes to 2 hours or more per administration. In addition, when the hydrogen gas-containing gas is administered through pulmonary administration by inhalation, breathing or the like, the hydrogen gas-containing gas can be administered in an environment at atmospheric pressure, or an environment, for example, at a high pressure within the range of above standard atmospheric pressure (about 1.013 atm) and not more than 7.0 atm, for example at a high pressure within the range of 1.02 to 7.0 atm, preferably 1.02 to 5.0 atm, more preferably 1.02 to 4.0 atm, still more preferably 1.02 to 1.35 atm to a human patient having Parkinson's disease, multiple system atrophy or progressive supranuclear palsy. By administration in an environment at a high pressure, systemic absorption of hydrogen in the human patient can be promoted.

The environment at a high pressure can be formed by use of a high-pressure housing (for example a capsule-shaped housing) designed to have a sufficient strength so that for example, the hydrogen gas-containing gas (for example hydrogen-containing oxygen or air) can be internally injected to produce a high pressure of above standard atmospheric pressure and not more than 7.0 atm in the housing. Preferably, the shape of the high-pressure housing is generally free from sharp edges and rounded because the housing has pressure resistance. Preferably, the material of the high-pressure housing has a small weight and a high strength, and examples of the material include reinforced plastics, carbon fiber composite materials, titanium alloys and aluminum alloys. In the high-pressure housing, the human patient having Parkinson's disease, multiple system atrophy or progressive supranuclear palsy can receive a composition for improving or preventing Parkinson's disease symptoms, the composition containing hydrogen gas together with oxygen gas or air.

In treatment of Parkinson's disease symptoms in Parkinson's disease, multiple system atrophy or progressive supranuclear palsy with the composition of the present invention, it is desirable to use a hydrogen gas producing apparatus, a hydrogen water producing apparatus or a hydrogen gas adding apparatus (for example the above-described hydrogen gas supplying apparatus (or gaseous hydrogen inhalation apparatus), a hydrogen adding device (or hydrogen water producing apparatus) or a non-destructive hydrogen incorporating device (apparatus for non-destructively dissolving hydrogen gas in a biologically applicable liquid such as a drip-feed solution encapsulated in a hydrogen-permeable bag) which has been confirmed to have a sufficient treatment effect and sufficient safety.

### EXAMPLES

The present invention will be described in further detail by way of Examples below, but the technical scope of the present invention is not limited to these Examples.

### Example 1

### Improvement of Parkinson's Disease Symptoms by Inhalation of Hydrogen Gas

### <Case 1>

A Parkinson's disease patient (67-year-old male, medical history: about 8 years) inhaled a mixed gas of hydrogen and air using a gaseous hydrogen inhalation apparatus (MHG-2000α (registered trademark); MiZ Company Limited) for about 90 minutes per day over about 2 months. Here, the mixed gas was inhaled every 2 or 3 days over first about 3 weeks and every day over subsequent about 5 weeks. The hydrogen concentration in MHG-2000α is about 6.0 to 7.0% by volume (hydrogen generation rate: about 140 ml/min). Before undergoing the inhalation of hydrogen gas, the patient had undergone drug therapy and rehabilitation, but had not exhibited a sign of improvement of Parkinson's disease symptoms (particularly, slow movement, shaking of limbs, forward-bending posture and the like).

As a result of inhaling the hydrogen gas over about 2 months, the patient improved to be almost comparable to a healthy person in complexion, voice, movement, gait and the like. In addition, when hydrogen gas was inhaled for 90 minutes for the first time, shaking of hands was stopped about 30 minutes after the start of inhalation, shaking under the jaw was stopped about 40 minutes after the start of inhalation, and the posture forward-bending in a dogleg shape was improved to a middle between the upright state and the dogleg shape state 90 minutes after the start of inhalation.

### <Case 2>

A Parkinson's disease patient (72-year-old male, medical history: about 6 years) inhaled a mixed gas of hydrogen and air using a gaseous hydrogen inhalation apparatus (MHG-2000α (registered trademark); MiZ Company Limited) for about 90 minutes or about 120 minutes per day. Here, the mixed gas was inhaled every day over about 4.5 months. The hydrogen concentration in MHG-2000α is about 6.0 to 7.0% by volume (hydrogen generation rate: about 140 ml/min).

In the patient, the waist had bent in a dogleg shape and the hands and the mouth had been considerably shaking before he underwent the inhalation of hydrogen gas for the first time. When hydrogen gas was inhaled for the first time, shaking of the hands was stopped about 45 minutes after the start of inhalation of hydrogen gas, shaking of the mouth was stopped about 55 minutes after the start of inhalation of hydrogen gas, and bending of the waist was improved about 90 minutes after the start of inhalation of hydrogen gas as compared to bending of the waist before the inhalation of hydrogen gas. In the patient, the hands and legs did not shake, or shook slightly, the mouth shook slightly, and the forward-bending posture was improved to a reasonably upright state, although the posture was not completely upright, immediately after the hydrogen gas was inhaled for about 90 minutes or about 120 minutes per day, substantially every day over about 1 month. The next day, however, the hands, the mouth and the legs were observed to shake often, but as described above, after inhalation of hydrogen gas there was no shaking or a little shaking, and it was observed that the symptoms tended to be significantly improved. Such a state was observed over about 3 months after the start of inhalation of hydrogen gas, and during this period, the patient also ingested hydrogen water (hydrogen concentration: 1.6 ppm) about 30 times. Marked improvement was observed about 4 months after the start of inhalation of hydrogen gas, shaking of the hands and the mouth was shaking that was too small to be recognized unless a careful observation is made, bending of the spine was reduced as compared to the symptom in the early stage, and the spine was straightened. In addition, the time taken for fastening buttons of clothing and the time taken for change of clothes were markedly shortened, it became possible to raise the arm, the body became flexible as a whole, and the voice became lively.

### <Case 3>

A patient (53-year-old female) with multiple system atrophy had symptoms very similar to those of Parkinson's disease, and unsteadily walked with support. After about 5 years after development of the disease, the disease was identified, and during those 5 years, the patient had taken 1.5 L of hydrogen water (hydrogen concentration: 1.6 ppm) produced by Aquela Blue (registered trademark) (Miraiplus Co., Ltd., Kanagawa, Japan) substantially every day. It is said that the average duration of life after development of multiple system atrophy is about 10 years, and a wheelchair is needed about 5 years after development of the disease. However, the symptoms of the patient were not so serious that a wheelchair was needed. This is presumed to be probably because the patient had ingested hydrogen water produced by Aquela Blue (registered trademark).

Soon after the disease was identified, the patient inhaled a mixed gas of hydrogen and air using a gaseous hydrogen inhalation apparatus (MHG-2000α (registered trademark); MiZ Company Limited) instead of ingesting hydrogen water. The hydrogen concentration in MHG-2000α is about 6.0 to 7.0% (hydrogen generation rate: about 140 ml/min). The patient constantly inhales hydrogen gas for about 1.5 hours to about 2 hours every day. In the early stage after the start of the inhalation, the patient became able to walk steadily and without support, and sometimes skip.

### <Case 4>

A Parkinson's disease patient (46-year-old male), who was affected about 3 years ago, took Pramipexol LA Tablet, 2.5 mg of FP Tablet OD and 100 mg of Carcopa Formulated Tablet L every day, and underwent glutathione infusion once every week. However, since symptoms specific to Parkinson's disease, such as slow movement, shaking of limbs and tiny step gait, were not sufficiently improved, hydrogen gas was inhaled using a hydrogen gas inhalation equipment (hydrogen concentration: about 4% by volume) for about 20 minutes every day over 3 months.

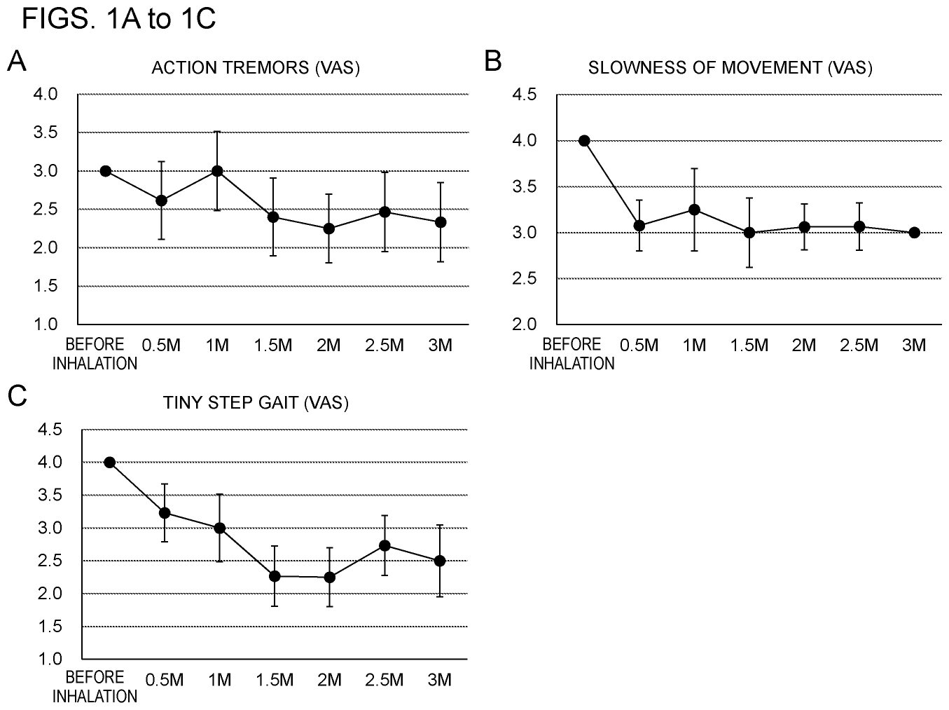

The symptoms of action tremors, slowness of movement and tiny step gait of the patient were measured with scale values of 0 to 5.0 (a greater value indicates a worse condition) on a visual analog scale (VAS). The results are shown in FIG. 1(A, B and C). After the start of the inhalation, it was observed that all of the symptoms were evidently improved as compared to the symptoms before the inhalation of hydrogen gas.

### <Case 5>

A Parkinson's disease patient (60-year-old male), who was affected about 4 years ago, took three drugs. The patient had such symptoms as slowness of movement, bending of the back in a dogleg shape, and shaking of a hand handling chopsticks. Thus, hydrogen gas was inhaled using a gaseous hydrogen inhalation apparatus (MHG-2000α (registered trademark); hydrogen concentration: about 6.0 to 7.0% by volume (hydrogen generation rate: about 140 ml/min)) for about 1 to 3 hours every day over 3 months.

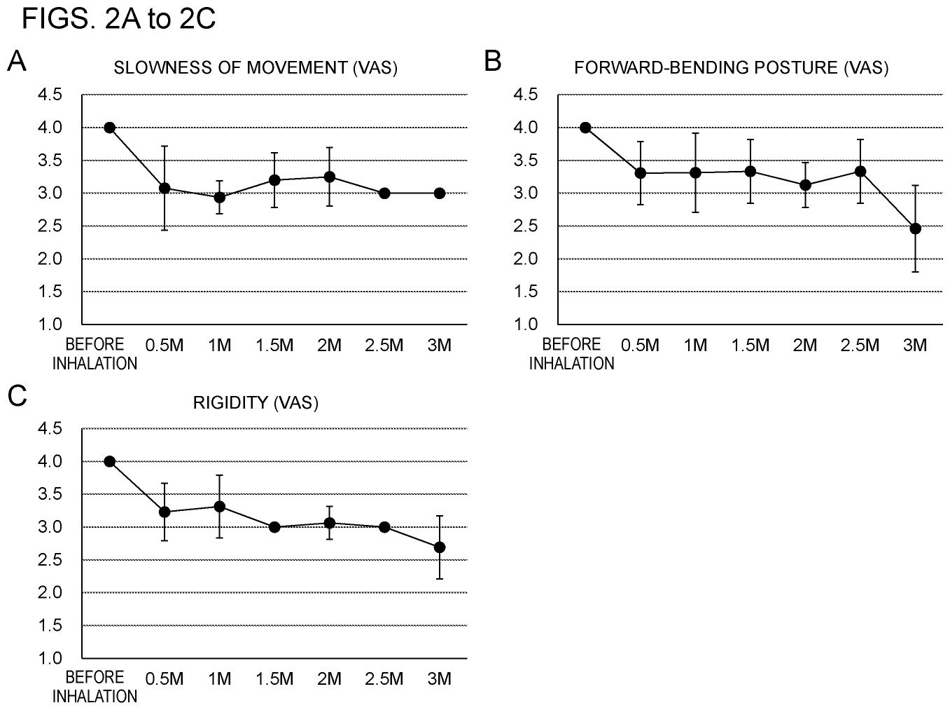

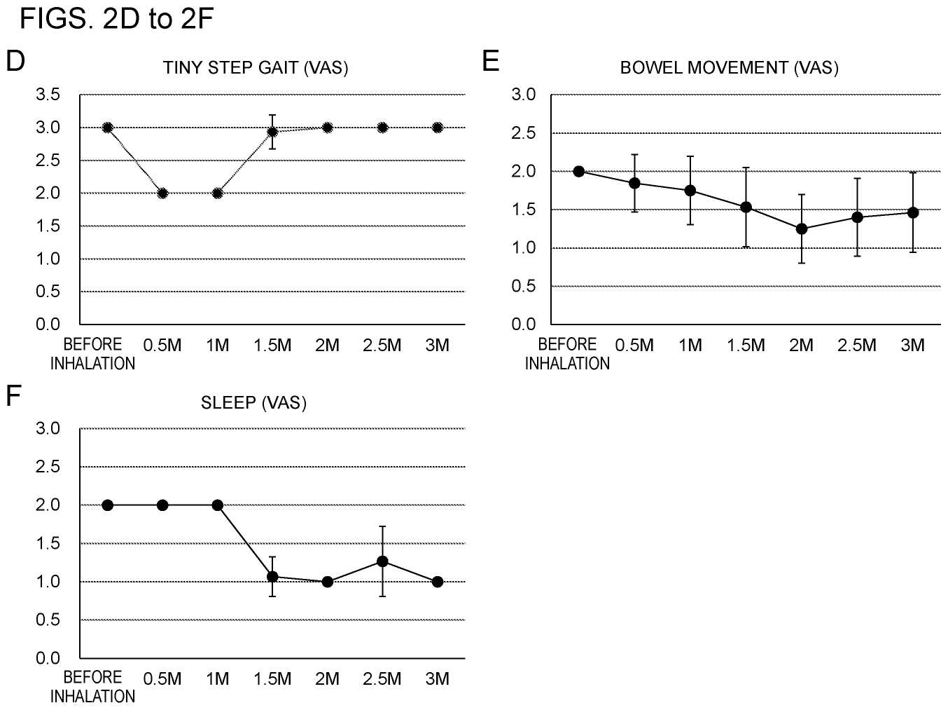

The symptoms of slowness of movement, forward-bending posture, rigidity, tiny step gait, bowel movement (constipation) and sleep (insomnia) of the patient were measured with scale values of 0 to 5.0 (a greater value indicates a worse condition) on a visual analog scale (VAS). The results are shown in FIG. 2 (A to F). After the start of the inhalation, it was observed that slowness of movement, forward-bending posture, rigidity, bowel movement (constipation) and sleep (insomnia) were evidently improved as compared to the symptoms before the inhalation of hydrogen gas.

### INDUSTRIAL APPLICABILITY

According to the present invention, Parkinson's disease symptoms, for example symptoms such as motor function disorders including tremors and/or forward-bending posture, or gait disorder can be improved or prevented only by administering hydrogen to a human patient having Parkinson's disease, multiple system atrophy or progressive supranuclear palsy. Hydrogen itself has no known side effect, and QOL of the patient can be markedly enhanced.

1. A composition for improving or preventing symptoms of human Parkinson's disease, multiple system atrophy or progressive supranuclear palsy, comprising a hydrogen gas-containing gas as an effective ingredient.

2. The composition according to claim 1, wherein the symptoms of human Parkinson's disease, multiple system atrophy or progressive supranuclear palsy are symptoms including at least tremors and/or forward-bending posture, or gait disorder.

3. The composition according to claim 1, wherein the multiple system atrophy is a disease selected from the group consisting of striatonigral degeneration, olivopontocerebellar atrophy and Shy-Drager syndrome.

4. The composition according to claim 1, wherein a hydrogen concentration of the hydrogen gas-containing gas is 0.5 to 18.5% by volume.

5. The composition according to claim 1, wherein the composition is administered to a patient by pulmonary administration.

6. The composition according to claim 1, wherein the composition is prepared in situ using a hydrogen gas producing apparatus in administration of the composition to the patient.

7. A method for improving or preventing symptoms of Parkinson's disease, multiple system atrophy or progressive supranuclear palsy in a human patient having Parkinson's disease, multiple system atrophy or progressive supranuclear palsy, the method comprising administering the composition according to claim 1 to the patient.

8. The method according to claim 7, wherein the symptoms of human Parkinson's disease, multiple system atrophy or progressive supranuclear palsy are symptoms including at least tremors and/or forward-bending posture, or gait disorder.

9. The method according to claim 7, wherein the multiple system atrophy is a disease selected from the group consisting of striatonigral degeneration, olivopontocerebellar atrophy and Shy-Drager syndrome.
